(19) United States
(12) Patent Application Publication
(10) Pub. No.: US 2005/0014236 A1
(43) Pub. Date: Jan. 20, 2005

(54) Method for producing L-arginine or L-lysine by fermentation

(76) Inventors: Yumi Matsuzaki, Kawasaki-shi (JP); Jun Nakamura, Kawasaki-shi (JP); Kenichi Hashiguchi, Kawasaki-shi (JP)

Correspondence Address: AJINOMOTO CORPORATE SERVICES, LLC;INTELLECTUAL PROPERTY DEPARTMENT, 1120 CONNECTICUT AVE., N.W., WASHINGTON, DC, 20036, US

(21) Appl. No.: 10/790,224

(22) Filed: Mar. 2, 2004

(30) Foreign Application Priority Data
Mar. 3, 2003 (JP) 2003-56129

(51) Int. Cl. C12P 13/08; C12N 9/10; C12N 1/21

(52) U.S. Cl. 435/115; 435/252.3; 435/193

(57) ABSTRACT

L-arginine or L-lysine is produced by culturing a coryneform bacterium having an L-arginine- or L-lysine-producing ability and modified so that glutamine synthetase activity is enhanced, e.g., a coryneform bacterium which is modified so that adenylylation of glutamine synthetase is eliminated. L-arginine or L-lysine are produced by culturing the bacterium in a medium and allowing L-arginine or L-lysine to accumulate in the medium, and collecting the L-arginine or L-lysine from the medium.

### BACKGROUND OF THE INVENTION

1. Field of the Invention

The present invention relates to a method for producing an L-amino acid, in particular, L-arginine and L-lysine, utilizing a coryneform bacterium. L-arginine is an industrially useful amino acid as an ingredient of liver function-promoting agents, amino acid infusions, comprehensive amino acid pharmaceuticals, and so forth. L-lysine is also an industrially useful amino acid as an additive for animal feeds, a component of health foods, amino acid infusions, and so forth.

2. Description of the Related Art

Breeding and improvement of microorganisms has been frequently used for production of an L-amino acid by fermentation. That is, since L-amino acid producing-abilities of wild-type strains per se are extremely low in many cases, imparting auxotrophy or analogue resistant mutation, imparting a metabolic regulation mutation, or utilizing a combination of these techniques are known. According to such methods, L-amino acids can be obtained with appropriate yields. However, in order to industrially produce L-amino acids at a low cost, it is necessary to further improve the fermentation yields.

In order to improve fermentation yields, it is preferable to control an activity of an enzyme in an amino acid biosynthetic pathway, i.e., to enhance a biosynthesis system producing an amino acid from a carbon source.

Basic amino acids have a particularly high nitrogen content. An arginine molecule comprises 6 carbon atoms and 4 nitrogen atoms, and a lysine molecule comprises 6 carbon atoms and 2 nitrogen atoms.

In amino acid fermentation, the nitrogen metabolism is as important as carbon metabolism, and it is important to modify nitrogen metabolism as well as carbon metabolism in order to improve fermentation yields. In the amino acid biosynthesis systems, a nitrogen atom is added by transamination of an amino group on glutamic acid. Therefore, an increase of intracellular concentrations of glutamine and glutamic acid will result in improvement of fermentation yields of amino acids.

Deletion of odhA gene encoding α-ketoglutarate dehydrogenase was reported as a method for increasing glutamic acid concentration (WO95/34672), and enhancement of glnA gene encoding glutamine synthetase was reported as a method for increasing glutamine concentration (European Patent Publication No. 1229121). Furthermore, it has been reported that elimination of the adenylylation of glutamine synthetase is effective as a method for enhancing the L-glutamine supplying pathway (European Patent Publication No. 1229121).

There have been no reports of the influence of the enhancement of biosynthesis of L-glutamine or L-glutamic acid on the productivity of L-arginine or L-lysine in coryneform bacteria.

### SUMMARY OF THE INVENTION

It is an object of the present invention to improve fermentation yields of L-amino acids, in particular, L-arginine and L-lysine, by modifying the nitrogen metabolism of coryneform bacteria.

It is a further object of the present invention to provide a coryneform bacterium which has an L-arginine- or L-lysine-producing ability, and wherein said bacterium is modified so that a glutamine synthetase activity is enhanced.

It is a further object of the present invention to provide the coryneform bacterium as described above, which comprises a modification which results in adenylylation of glutamine synthetase being reduced or eliminated.

It is a further object of the present invention to provide the coryneform bacterium as described above, wherein said modification is selected from the group consisting of
a) mutating the adenylylation site of glutamine synthetase;
b) intracellular activity of glutamine synthetase adenylyltransferase is reduced,
c) intracellular activity of PII protein is reduced, and
d) a nitrogen metabolism regulation protein is modified so that intracellular activity of glutamine synthetase is increased.

It is a further object of the present invention to provide the coryneform bacterium as described above, wherein said adenylylation site comprises position 405 of SEQ ID NO: 20 and wherein said mutation comprises replacement of the wild-type tyrosine residue with another amino acid.

It is a further object of the present invention to provide the coryneform bacterium as described above, wherein a gene encoding the glutamine synthetase adenylyltransferase on a chromosome of said bacterium is disrupted.

It is a yet further object of the present invention to provide the coryneform bacterium as described above, wherein the nitrogen metabolism regulation protein is an amtR gene product which does not function normally.

It is a still further object of the present invention to provide the coryneform bacterium as described above, wherein the amtR gene on a chromosome of said bacterium is disrupted.

It is a further object of the present invention to provide the coryneform bacterium as described above, which is modified so that an arginine repressor does not function normally.

It is a futher object of the present invention to provide the coryneform bacterium as described above, wherein a gene on a chromosome of said bacterium encoding the arginine repressor is disrupted.

It is a futher object of the present invention to provide a method for producing L-arginine or L-lysine comprising
a) culturing the coryneform bacterium as described above in a medium,
b) allowing L-arginine or L-lysine to accumulate in the medium, and
c) collecting the L-arginine or L-lysine from the medium.

According to the present invention, in the production of L-arginine or L-lysine by fermentation using a coryneform bacterium, fermentation yield of L-arginine or L-lysine can be increased by modifying nitrogen metabolism regulation. Moreover, the DNA of the present invention can be used for breeding of L-arginine or L-lysine producing coryneform bacteria.

### DESCRIPTION OF THE PREFERRED EMBODIMENTS

The inventors of the present invention conducted research to achieve the aforementioned object. As a result, a strain of coryneform bacterium was found in which nitrogen metabolism regulating mechanism, including the adenylylation of glutamine synthetase, was modified, and showed superior fermentative production of amino acids, in particular, L-arginine and L-lysine, due to an increase of intracellular concentrations of glutamine and glutamic acid.

Hereinafter, the present invention will be explained in detail.

Coryneform Bacterium Used for Construction of the Bacterium of the Present Invention

In the present invention, coryneform bacteria include bacteria having been hitherto classified into the genus Brevibacterium, but united into the genus Corynebacterium at present (Int. J. Syst. Bacteriol., 41, 255 (1981)), and include bacteria belonging to the genus Brevibacterium closely relative to the genus Corynebacterium. Examples of such coryneform bacteria are listed below:
Corynebacterium acetoacidophilum
Corynebacterium acetoglutamicum
Corynebacterium alkanolyticum
Corynebacterium callunae
Corynebacterium glutamicum
Corynebacterium lilium
Corynebacterium melassecola
Corynebacterium thermoaminogenes
Corynebacterium herculis
Brevibacterium divaricatum
Brevibacterium flavum
Brevibacterium immariophilum
Brevibacterium lactofermentum
Brevibacterium roseum
Brevibacterium saccharolyticum
Brevibacterium thiogenitalis
Corynebacterium ammoniagens
Brevibacterium album
Brevibacterium cerinum
Microbacterium ammoniaphilum
   Specifically, the following strains of bacteria are exemplified:
Corynebacterium acetoacidophilum ATCC 13870
Corynebacterium acetoglutamicum ATCC 15806
Corynebacterium alkanolyticum ATCC21511
Corynebacterium callunae ATCC15991
Corynebacterium glutamicum ATCC13020, ATCC 13032, ATCC13060
Corynebacterium lilium ATCC 15990
Corynebacterium melassecola ATCC 17965
Corynebacterium efficiens AJ12340 (FERM BP-1539)
Corynebacterium herculis ATCC13868
Brevibacterium divaricatum ATCC14020
Brevibacterium flavum ATCC 13826, ATCC 14067, AJ 12418(FERM BP-2205)
Brevibacterium immariophilum ATCC14068
Brevibacterium lactofermentum ATCC 13869
Brevibacterium roseum ATCC13825
Brevibacterium saccharolyticum ATCC14066 Brevibacterium thiogenitalis ATCC 19240
Corynebacterium ammoniagenes ATCC6871, ATCC6872
Brevibacterium album ATCC 15111
Brevibacterium cerinum ATCC 15112
Microbacterium ammoniaphilum ATCC 15354

These strains can be obtained from, for example, the American Type Culture Collection (10801 University Boulevard, Manassas, Va. 20110-2209, United States of America). That is, each strain is assigned a registration number, and one can request provision of a strain by its registration number. The registration numbers corresponding to the strains are indicated in the catalog of the American Type Culture Collection.

Furthermore, the AJ12340 strain was deposited on Oct. 27, 1987 at the National Institute of Bioscience and Human-Technology, Agency of Industrial Science and Technology, Ministry of International Trade and Industry (currently, the independent administrative corporation, National Institute of Advanced Industrial Science and Technology, International Patent Organism Depositary (Chuo Dai-6,1-1 Higashi 1-Chome, Tsukuba-shi, Ibaraki-ken, Japan, postal code: 305-5466)) as an international deposit under the provisions of the Budapest Treaty, and received an accession number of FERM BP-1539. The AJ12418 strain was deposited on Jan. 5, 1989 at the National Institute of Bioscience and Human-Technology, Agency of Industrial Science and Technology, Ministry of International Trade and Industry as an international deposit under the provisions of the Budapest Treaty and received an accession number of FERM BP-2205.

In the present invention, the term “L-arginine-producing ability” means an ability to cause accumulation of L-arginine in a medium when the bacterium is cultured in the medium. This L-arginine-producing ability may be a property of a wild-type coryneform bacterium strain, or a property imparted or enhanced by breeding.

In the present invention, the term “L-lysine-producing ability” means an ability to cause accumulation of L-lysine in a medium when the bacterium is cultured in the medium. This L-lysine-producing ability may be a property of a wild-type coryneform bacterium strain, or a property imparted or enhanced by breeding.

A corynefrom bacterium having L-lysine- or L-arginine-producing ability can be obtained by imparting an L-lysine- or L-arginine-producing ability to a wild-type strain of a corynefrom bacterium. Methods conventionally used for breeding of coryneform bacteria, Escherichia bacteria and so forth, can be used to impart the L-lysine- or L-arginine-producing ability. For example, such methods include, but are not limited to acquisition of auxotrophic mutant strains, analogue-resistant strains or metabolic regulation mutant strains, creation of recombinant strains in which an L-lysine or L-arginine biosynthesis system enzyme is enhanced (see “Amino Acid Fermentation”, the Japan Scientific Societies Press [Gakkai Shuppan Center], 1st Edition, published on May 30, 1986, pp. 77 to 100), and so forth. Properties of auxotrophy, analogue resistance, metabolic regulation mutation, and so forth, may be individually imparted or two or more may be imparted in combination when breeding L-lysine- or L-arginine-producing bacteria. The biosynthesis system enzyme may be individually enhanced or two or more of them may be enhanced in combination. Furthermore, the impartation of properties including auxotrophy, analogue resistance, metabolic regulation mutation, and so forth, may be combined with the enhancement of biosynthesis system enzyme.

L-arginine-producing coryneform bacteria are not limited so long as they have an ability to produce L-arginine. Examples of such coryneform bacteia include, but are not limited to, a bacterium which is resistant to a certain agent, for example, a sulfa drug, 2-thiazolealanine, α-amino-β-hydroxyvaleric acid, or the like; which is auxotrophic for L-histidine, L-proline, L-threonine, L-isoleucine, L-methionine, or L-tryptophan, in addition to resistant to 2-thiazolealanine (Japanese Patent Laid-open No. 54-44096); which is resistant to ketomalonic acid, fluoromalonic acid or monofluoroacetic acid (Japanese Patent Laid-open No. 57-18989), which is resistant to argininol (Japanese Patent Laid-open No. 62-24075); which is resistant to X-guanidine (X represents a derivative of fatty acid or aliphatic chain, Japanese Patent Laid-open No. 2-186995).

The coryneform bacterium producing L-arginine can be bred to be resistant to 5-azauracil, 6-azauracil, 2-thiouracil, 5-fluorouracil, 5-bromouracil, 5-azacytosine, 6-azacytosine, and so forth; to be resistant to arginine hydroxamate and 2-thiouracil; to be resistant to arginine hydroxamate and 6-azauracil (see Japanese Patent Laid-open No. 49-126819); to be resistant to a histidine analogue or tryptophan analogue (see Japanese Patent Laid-open No. 52-114092); to be auxotrophic for at least one of methionine, histidine, threonine, proline, isoleucine, lysine, adenine, guanine and uracil (or uracil precursor) (see Japanese Patent Laid-open No. 52-99289); to be resistant to arginine hydroxarate (see Japanese Patent Publication No. 51-6754); to be auxotrophic for succinic acid or resistant to a nucleic acid base analogue (see Japanese Patent Laid-open No. 58-9692); to be unable to metabolize arginine and to be resistant to an arginine antagonist and canavanine and to be auxotorophic for lysine (see Japanese Patent Laid-open No. 52-8729); to be resistant to arginine, arginine hydroxamate, homoarginine, D-arginine and canavanine, or to be resistant to arginine hydroxamate and 6-azauracil (see Japanese Patent Laid-open No. 53-143288); to be resistant to canavanine (see Japanese Patent Laid-open No. 53-3586) and so forth.

Specific examples of L-arginine producing coryneform bacteria include the following strains.
Brevibacterium flavum AJ 11169 (FERM P-4161)
Brevibacterium lactofermentum AJ 12092 (FERM P-7273)
Brevibacterium flavum AJ11336 (FERM P-4939)
Brevibacterium flavum AJ 11345 (FERM P-4948)
Brevibacterium lactofermentum AJ12430 (FERM BP-2228)

The AJ11169 strain and AJ12092 strain are 2-thiazolealanine resistant strains described in Japanese Patent Laid-open No. 54-44096, the AJ 11336 strain is a strain having argininol resistance and sulfadiazine resistance described in Japanese Patent Publication (KOKOKU) No. No. 62-24075, the AJ11345 strain is a strain having argininol resistance, 2-thiazolealanine resistance, sulfaguanidine resistance and histidine auxotrophy described in Japanese Patent Publication No. No. 62-24075, and the AJ12430 strain is a strain having octylguanidine resistance and 2-thiazolealanine resistance described in Japanese Patent Laid-open No. 2-186995.

AJ11169 was deposited on Aug. 3, 1977 at the National Institute of Bioscience and Human-Technology, Agency of Industrial Science and Technology, Ministry of International Trade and Industry (currently, the independent administrative corporation, National Institute of Advanced Industrial Science and Technology, International Patent Organism Depositary (Chuo Dai-6,1-1 Higashi 1-Chome, Tsukuba-shi, Ibaraki-ken, Japan, postal code: 305-5466) and given an accession number of FERM P-4161. Then, it was converted to an international deposit based on the Budapest Treaty on Sep. 27, 1999 and given an accession number of FERM BP-6892.

AJ12092 was deposited on Sep. 29, 1983 at the National Institute of Bioscience and Human-Technology, Agency of Industrial Science and Technology, Ministry of International Trade and Industry and given an accession number of FERM P-7273. Then, it was converted to an international deposit based on the Budapest Treaty on Oct. 1, 1999 and given an accession number of FERM BP-6906.

AJ11336 was deposited on Apr. 25, 1979 at the National Institute of Bioscience and Human-Technology, Agency of Industrial Science and Technology, Ministry of International Trade and Industry and given an accession number of FERM P-4939. Then, it was converted to an international deposit based on the Budapest Treaty on Sep. 27, 1999 and given an accession number of FERM BP-6893.

AJ11345 was deposited on Apr. 25, 1979 at the National Institute of Bioscience and Human-Technology, Agency of Industrial Science and Technology, Ministry of International Trade and Industry and given an accession number of FERM P-4948. Then, it was converted to an international deposit based on the Budapest Treaty on Sep. 27, 1999 and given an accession number of FERM BP-6894.

AJ12430 was deposited on Dec. 26, 1988 the National Institute of Bioscience and Human-Technology, Agency of Industrial Science and Technology, Ministry of International Trade and Industry as an international deposit based on the Budapest Treaty, and given an accession number of FERM BP-2228.

Breeding to impart or enhance L-lysine productivity can be performed by introducing one or more mutations as follows. Such artificial mutants are as follows: S-(2-aminoethyl)-cysteine (hereinafter referred to as “AEC”) resistant mutants; mutants requiring amino acids such as L-homoserine for their growth (see Japanese Patent Publication Nos. 4828078 and 566499); mutants resistant to AEC and requiring amino acids such as L-leucine, L-homoserine, L-proline, L-serine, L-arginine, L-alanine, L-valine, etc. (see U.S. Pat. Nos. 3,708,395 and 3,825,472); L-lysine-producing mutants resistant to DL-α-amino-β-caprolactam, α-aminolauryllactam, aspartic acid analogues, sulfa drugs, quinoids and N-lauroylleucine, and L-lysine-producing mutants resistant to oxaloacetate decarboxylase or respiratory system enzyme inhibitors (see Japanese Patent Laid-Open Nos. 5053588, 5031093, 52102498, 539394, 5386089, 559783, 559759, 5632995, 5639778, Japanese Patent Publication Nos. 5343591, 531833); L-lysine-producing mutants requiring inositol or acetic acid (see Japanese Patent Laid-Open Nos. 559784, 568692); L-lysine-producing mutants sensitive to fluoropyruvic acid or to temperatures of 34° C. or higher (see Japanese Patent Laid-Open No. 5386090); L-lysine-producing mutants of bacteria belonging to the genus Brevibacterium or Corynebacterium resistant to ethylene glycol (see U.S. Pat. No. 4,411,997).

Hereinafter, methods for imparting or enhancing L-amino acid-producing ability by enhancing an L-arginine or L-lysine biosynthesis system enzyme activity are exemplified.

Enhancement of enzyme activity can be attained by introducing a mutation into a gene encoding the enzyme so that intracellular activity of the enzyme is enhanced, or by using a genetic recombination technique utilizing the gene.

The enzyme in L-arginine biosynthetic pathway may be one or more kinds of enzymes selected from N-acetylglutamyl phosphate reductase (argC), omithine acetyltransferase (argJ), N-acetylglutamate kinase (argB), acetylornithine transaminase (argD), ornithine carbamoyltransferase (argF), argininosuccinate synthase (argG) and argininosucciniate lyase (argH), carbamoyl phosphate synthetase (carAB). The designations of the genes encoding these enzymes are indicated in the parentheses after the name of the enzymes, respectively.

Examples of the mutation that increases the activity of the protein encoded by the genes as described above include a mutation of a promoter sequence that increases transcription amount of the gene or a mutation in the coding region of the gene that increases specific activity of the enzyme protein and so forth.

Furthermore, enhancement of the enzymatic activity using a genetic recombination technique can be attained by, for example, increasing the copy number of a gene encoding the enzyme in a cell. For example, a DNA fragment containing the gene can be ligated to a vector functioning in a microorganism, preferably a multi-copy type vector, to prepare recombinant DNA and used to transform the microorganism.

Other than the aforementioned gene amplification, the enhancement of gene expression can also be attained by replacing an expression regulatory sequence, such as a promoter of the gene, in a chromosomal DNA or plasmid with a stronger one (WO00/18935). Examples of strong promoters include lac promoter, trp promoter, trc promoter, and so forth. Furthermore, by introducing nucleotide substitution, or the like, into the promoter region of the lysE gene, the promoter can be modified so as to become a stronger promoter. The substitution or modification of the promoter enhances expression of the gene. These modifications of the expression regulatory sequence can be combined with the increase of the copy number of the gene.

Examples of the L-lysine biosynthesis system gene include, for example, a gene encoding the aspartokinase α-subunit protein or β-subunit protein for which synergistic feedback inhibition by L-lysine and L-threonine is desensitized (International Patent Publication WO94/25605), wild-type phosphoenolpyruvate carboxylase gene derived from coryneform bacterium (Japanese Patent Laid-open No. 60-87788), gene encoding wild type dihydrodipicolinate synthetase derived from coryneform bacterium (Japanese Patent Publication No. 6-55149), and so forth.

The aforementioned methods for enhancing an activity of L-arginine biosynthesis system enzyme can be similarly applied to an L-lysine biosynthesis system.

Furthermore, by modifying a coryneform bacterium so that an arginine repressor does not function normally in the cell of the bacterium, an L-lysine- or L-arginine-producing ability can be imparted or enhanced.

Expression of an arginine biosynthesis system gene such as a gene of N-acetylglutamate synthase usually suffers marked supression by arginine in the medium. As for coryneform bacteria, it has been determined that production of some enzymes of the L-arginine biosynthesis system is suppressed by L-arginine. Furthermore, it was reported that, while some L-arginine biosynthesis system enzymes were suppressed by L-arginine, the suppression of these enzymes by L-arginine was eliminated in mutant strains of coryneform bacteria showing improved L-arginine accumulation (Agric. Biol. Chem., 43(1), 105, 1979).

Furthermore, as for Escherichia coli, a repressor of L-arginine biosynthesis system (arginine repressor) and a gene encoding the repressor were identified (Proc. Natl. Acad. Sci. U.S.A. (1987), 84(19), 6697-701), and binding interactions of the repressor protein and various L-arginine biosynthesis system genes were also investigated (Proc. Natl. Acad. Sci. U.S.A. (1987), 84(19), 6697-701; J. Mol. Biol. (1992), 226, 367-386).

Arginine is produced through a biosynthetic pathway peculiar to arginine via intermediates such as omithine and citrulline, and carbamylphosphate is taken up in this pathway. Therefore, it is considered necessary to enhance the carbamylphosphate synthesis pathway in order to increase the fermentation yield of arginine.

Carbamylphosphate is produced from a carbonate ion, glutamine and ATP. In coryneform bacteria, the carbonate ion is derived from culture broth, and ATP is generated in a metabolic process of saccharide. Therefore, supply of glutamine is important for production of carbamylphosphate.

Based on the above, the L-lysine- and L-arginine-producing abilities can be further improved by combining the inactivation of the arginine repressor technique with the increase of the glutamine synthetase activity technique, described later.

In the present invention, the “arginine repressor” refers to a protein that has an effect of suppressing the L-arginine biosynthesis, and if expression amounts of a gene that encodes this protein increases in a microorganism, L-arginine-producing ability will be reduced. If the expression amount decreases or the protein disappears, the L-arginine-producing ability will be improved. Hereinafter, a gene encoding the arginine repressor is called argR gene. The expression “arginine repressor does not function normally” means that the activity of the arginine repressor is reduced or eliminated as compared with a wild-type strain or unmodified strain.

The nucleotide sequence of the argR gene of the Brevibacterium flavum and the amino acid encoded thereby are shown in SEQ ID NO: 15, and the amino acid sequence is shown in SEQ ID NO: 16.

In the present invention, the arginine repressor, which is an object for activity reduction, may have an amino acid sequence which includes deletion, substitution, insertion or addition of one or more amino acid residues in the aforementioned amino acid sequence of the arginine repressor at one or more sites.

The number of “one or more” amino acid residues varies depending on the position of the amino acid residues in the three-dimensional structure of the protein and type of the amino acids. This is for the following reason. That is, some amino acids are highly homologous to one another, and the difference between such amino acids does not significantly influence the three-dimensional structure of the protein. Thus, the mutant arginine repressor of the present invention may be a protein exhibiting 30 to 50% or more, preferably 50 to 70% or more, more preferably 80 to 90% or more, most preferably 95% or more, of homology with respect to the whole amino acid residues constituting the arginine repressor and having the arginine repressor activity.

The DNA encoding a protein substantially identical to the aforementioned arginine repressor includes a DNA that is hybridizable with the argR gene under stringent conditions and encodes a protein having the arginine repressor activity. “Stringent conditions” are conditions under which a so-called specific hybrid is formed, and a non-specific hybrid is not formed. It is difficult to clearly express this condition by using any numerical value. However, for example, the stringent conditions include a condition whereby DNA species having high homology, for example, DNAs having homology of 50% or more, preferably 70% or more, more preferably 80% or more, most preferably 90% or more, hybridize with each other, but DNAs having homology lower than the above do not hybridize with each other. Alternatively, stringent conditions include conditions whereby DNAs hybridize with each other at a salt concentration corresponding to typical conditions of Southern hybridization, i.e., approximately 1×SSC, 0.1% SDS, and preferably 0.1×SSC, 0.1% SDS, at 60° C.

Examples of a method for reducing the activity of arginine repressor of coryneform bacteria include, for example, treating a coryneform bacterium with ultraviolet ray irradiation or a mutagenesis agent used in a usual mutagenesis treatment such as N-methyl-N′-nitro-N-nitrosoguanidine (NTG) or nitrous acid and selecting a mutant strain showing reduced activity of arginine repressor. Other than the mutation treatment, a coryneform bacterium having reduced arginine repressor activity can also be obtained by transforming a coryneform bacterium with a DNA containing the argR gene encoding the modified arginine repressor, so as not to produce arginine repressor that normally functions (deletion-type argR gene) by deleting a partial sequence of the argR gene to cause recombination between the deletion-type argR gene and the argR gene on a chromosome and thereby disrupt the argR gene on a chromosome.

The gene disruption concerning the argR gene can be carried out in the same manner as that for the gene disruption for the glnE gene described later.

The origin of the argR gene is not particularly limited, so long as it has homology to such a degree that it causes homologous recombination with the argR gene of a target microorganism. Specifically, examples of the argR genes of coryneform bacteria include, but are not limited to, the argR gene of the Brevibacterium flavum described above and the argR gene of Corynebacterium glutamicum (GenBank accession AF049897). These argR genes are highly homologous, and therefore it is considered that an argR gene from any homologous genus or species may also be used for gene disruption.

Construction of Coryneform Bacterium of the Present Invention

The coryneform bacterium of the present invention has an L-lysine- or L-arginine-producing ability as described above, and which is modified so that the intracellular glutamine synthetase (henceforth also referred to as “GS”) activity is enhanced.

In breeding of the coryneform bacterium of the present invention, either the impartation of the L-lysine- or L-arginine-producing ability or the enhancement of the GS activity may be carried out first.

The expression “modified so that the intracellular GS activity is enhanced” means that the GS activity per cell has become higher than that of an unmodified strain such as a wild-type coryneform bacterium. For example, those in which the number of GS molecules per cell is increased, those in which the GS activity per GS molecule is increased, and so forth, are encompassed. The “GS activity” means an activity of catalyzing a reaction of producing glutamine from glutamic acid and ammonia using ATP. Furthermore, the wild-type coryneform bacterium that serves as an object for comparison may be, for example, the Brevibacterium lactofermentum ATCC 13869.

Specifically, the coryneform bacterium which has enhanced GS activity is preferably, for example, a coryneform bacterium exhibiting a GS activity of 100 to 150 nmol/min/mg of intracellular protein or more, or a coryneform bacterium exhibiting a GS activity of 2 to 3 times higher than that of a wild-type strain. However, the coryneform bacterium of the present invention is not limited to these. The GS activity can be measured by the method described in Journal of Fermentation and Bioengineering, Vol. 70, No. 3, 182-184, 1990. Furthermore, intracellular protein can be quantified by, for example, using Protein Assay (Bio-Rad) with bovine serum albumin standard.

Enhancing the intracellular glutamine synthetase activity can be attained by, for example, enhancing expression of a gene encoding GS. Enhancing the expression of the GS gene can be attained by increasing the copy number of the gene encoding GS. For example, a gene fragment encoding GS is ligated to a vector functioning in the bacterium, preferably a multi-copy type vector, to prepare recombinant DNA and used to transform a coryneform bacterium.

Any GS genes derived from coryneform bacteria or derived from other organisms such as Escherichia bacterium may be used. Among these, genes derived from coryneform bacteria are preferred for their ease of expression.

The genesglnA (FEMS Microbiology Letters, 81-88 (154), 1997) andglnA2 (EP1229121, L. Nolden et al., FEMS Microbiology Letters, 201 (2001) 91-98) have been reported as genes encoding GS of coryneform bacteria.

The nucleotide sequence of the glnA gene and the amino acid sequence encoded by the gene of Brevibacterium lactofermentum are shown in SEQ ID NO: 19, and the amino acid sequence is shown in SEQ ID NO: 20. Although the amino acid encoded by the initiation codon (nucleotide numbers 1 to 3) is shown as valine in SEQ ID NOS: 19 and 20, it is most likely methionine. The nucleotide sequence of glnA2 gene and the amino acid sequence encoded thereby are described in Japanese Patent Laid-open No. 2002-300887 EP1229121.

In the present invention, GS may include deletion, substitution, insertion or addition of one or more amino acid residues at one or more sites so long as the GS activity is not reduced. The number of “one or more” amino acid residues varies depending on the position of the amino acid residues in the three-dimensional structure of the protein and the type of amino acid. However, the mutant GS of the present invention may be a protein exhibiting 30 to 50% or more, preferably 50 to 70% or more, more preferably 80 to 90% or more, most preferably 95% or more, homology with respect to the whole amino acid residues constituting GS and having the GS activity.

The DNA encoding a protein substantially identical to the aforementioned GS includes a DNA that is hybridizable with the glnA or glnA2 gene under stringent conditions and encodes a protein having the GS activity. The stringent conditions include conditions whereby DNAs having high homology, for example, DNAs having homology of 50% or more, preferably 70% or more, more preferably 80% or more, most preferably 90% or more, hybridize with each other, but DNAs having homology lower than the above do not hybridize with each other. Alternatively, the stringent conditions include conditions whereby DNAs hybridize with each other at a salt concentrations corresponding to typical washing conditions of Southern hybridization, i.e., approximately 1×SSC, 0.1% SDS, preferably 0.1×SSC, 0.1% SDS, at 60° C.

Furthermore, the intracellular glutamine synthetase activity of coryneform bacterium can also be enhanced by modifying the bacterium so that glutamine synthetase adenylylation is reduced or eliminated.

GS changes into an inactive form by adenylylation of a tyrosine residue in its amino acid sequence (Proc. Natl. Acad. Sci. USA, 642-649, (58) 1967; J. Biol. Chem., 3769-3771, (243) 1968). Therefore, the intracellular GS activity can be enhanced by reducing or eliminating the adenylylation of GS. The reduction or elimination of adenylylation referred to herein means not only substantially complete elimination of the adenylylation, but also a reduction of the adenylylation so that the intracellular GS activity is enhanced. The “reduction” means that the adenylylation of GS is reduced compared with that of a wild-type strain or unmodified strain of a coryneform bacterium. The wild-type coryneform bacterium that serves as an object for comparison may be, for example, the Brevibacterium lactofermentum ATCC 13869.

Hereinafter, means for reducing or eliminating the adenylylation of GS are exemplified.

(1) Modification of the Adenylylation Site of Glutamine Synthetase

In Escherichia coli etc., the adenylylation of GS is generally performed by glutamine synthetase adenylyltransferase (henceforth also referred to as “ATase”, Proc. Natl. Acad. Sci. USA, 1703-1710, (58)1967). Also in coryneform bacteria, GS is converted into its inactive form by adenylylation with glutamine synthetase adenylyltransferase (FEMS Microbiology Letters, 303-310, (173) 1999; FEMS Microbiology Letters, 201 (2001) 91-98). It has been suggested that, in coryneform bacteria, the tyrosine residue at position 405 of the glnA gene product (Genebank accession Y13221) is adenylylated (FEMS Microbiology Letters, 303-310, (173) 1999). This inactivation of GS by the adenylylation of GS can be eliminated by introducing a mutation into the glnA gene so that the tyrosine residue is replaced with another amino acid residue such as a phenylalanine residue.

Furthermore, it is expected that the aforementioned glnA2 product also suffers from adenylylation, with similar results, like the glnA product. Therefore, the inactivation by adenylylation of GS encoded by glnA2 can be reduced or eliminated by introducing a mutation into the glnA2 gene so that the tyrosine residue corresponding to tyrosine residue at position 405 of GlNA is replaced with another amino acid residue, such as a phenylalanine residue.

A DNA encoding GS whereby adenylylation is reduced or eliminated can be obtained by modifying the sequence of glnA or glnA2 so that the sequence includes a mutation resulting in elimination of adenylylation of the encoded GS. The obtained mutant gene can be introduced into a coryneform bacterium by any means similar to that used for the enhancement of L-arginine biosynthesis enzyme gene described above.

GS having a modified adenylylation site may also have an amino acid sequence including deletion, substitution, insertion or addition of one or more amino acid residues at one or more sites in addition to the mutation reducing or eliminating adenylylation of GS, so long as the GS activity is not reduced.

(2) Reduction of Glutamine Synthetase Adenylyltransferase Activity

Adenylylation of GS can also be reduced or eliminated by reducing the activity of intracellular glutamine synthetase adenylyltransferase (ATase). The reduction of the ATase activity referred to herein means not only complete elimination of the activity but also such reduction of the activity that the intracellular ATase activity is reduced compared with that of a wild-type strain or unmodified strain of a coryneform bacterium. For example, those in which the copy number of ATase molecules per cell is reduced, those in which the ATase specific activity per ATase molecule is reduced and so forth are encompassed. Examples of the wild-type coryneform bacterium that serves as an object for comparison include, for example, the Brevibacterium lactofermentum ATCC 13869.

The gene glnE of the Brevibacterium lactofermentum ATCC 13869 strain has been elucidated as a gene encoding ATase (EP1229121). The nucleotide sequence of the gene and the amino acid encoded thereby are shown in SEQ ID NO: 17, and the amino acid sequence is shown in SEQ ID NO: 18.

In the present invention, ATase, which is the object of the activity reduction, may have an amino acid sequence including deletion, substitution, insertion or addition of one or more amino acid residues in the aforementioned amino acid sequence of ATase at one or more sites.

The number of “one or more” amino acid residues varies depending on the position of the amino acid residues in the three-dimensional structure of the protein and type of amino acids. However, the mutant ATase of the present invention may be a protein exhibiting 30 to 50% or more, preferably 50 to 70% or more, more preferably 80 to 90% or more, most preferably 95% or more, homology with respect to the whole amino acid residues constituting Atase, and having the ATase activity.

The DNA encoding a protein substantially identical to the aforementioned ATase includes a DNA that is hybridizable with the glnE gene under stringent conditions and codes for a protein having the ATase activity. “Stringent conditions” include conditions whereby DNAs having high homology, for example, DNAs having homology of 50% or more, preferably 70% or more, more preferably 80% or more, most preferably 90% or more, hybridize with each other, but DNAs having homology lower than the above do not hybridize with each other. Alternatively, stringent conditions include conditions whereby DNAs hybridize with each other at a salt concentration corresponding to typical washing conditions of Southern hybridization, i.e., approximately 1×SSC, 0.1% SDS, preferably 0.1×SSC, 0.1% SDS, at 60° C.

Examples of the method for reducing the intracellular activity of ATase of coryneform bacteria include, for example, treating a coryneform bacterium with ultraviolet ray irradiation or a mutagenesis agent used in a usual mutagenesis treatment such as N-methyl-N′-nitro-N-nitrosoguanidine (NTG) or nitrous acid and selecting a mutant strain showing reduced activity of ATase. Besides the mutation treatment, a coryneform bacterium showing reduced activity of ATase can also be obtained by transforming a coryneform bacterium with a DNA containing the gene encoding ATase (glnE) modified so as not to produce ATase that functions normally (deletion-type glnE gene) by deleting a part of the glnE gene to cause recombination between the deletion-type glnE gene and the glnE gene on a chromosome and thereby disrupt the glnE gene on a chromosome. Such gene destruction by gene substitution utilizing homologous recombination has been established, and there are methods of utilizing a linear DNA, a plasmid that contains a temperature sensitive replication origin and so forth.

A glnE gene on a host chromosome can be replaced with the deletion-type glnE gene, for example, as follows. That is, a recombinant DNA is prepared by inserting a temperature sensitive replication origin, a mutant glnE gene and a marker gene for resistance to a drug such as chloramphenicol, and used to transform a coryneform bacterium. Furthermore, the resultant transformant strain is cultured at a temperature at which the temperature sensitive replication origin does not function, and then the transformant strain can be cultured in a medium containing the drug to obtain a transformant strain in which the recombinant DNA is incorporated into a chromosomal DNA.

In such a strain in which the recombinant DNA is incorporated into a chromosomal DNA as described above, the mutant glnE gene is recombined with the glnE gene originally present on a chromosome, and the two fusion genes chromosomal glnE gene and deletion-type glnE gene are inserted into the chromosome so that the other portions of the recombinant DNA (vector segment, temperature sensitive replication origin and drug resistance marker) are present between the two fusion genes.

In the glnE ligated to the plasmid, an internal sequence not containing any promoter and initiation codon is used. Therefore, the structural gene of GlNE is interrupted where the other portions of the recombinant DNA (vector segment, temperature sensitive replication origin and drug resistance marker) are present between the two fusion genes, and hence GlnE loses the function.

Furthermore, glnE having a deleted internal sequence may also be used as the deletion-type glnE. In such a case, the native glnE gene is dominant in a state that two of fusion genes of the chromosomal glnE and the deletion-type glnE are inserted into the chromosomal DNA, and therefore the transformant strain expresses normal ATase. Then, in order to leave only the deletion-type glnE gene on the chromosomal DNA, one copy of the glnE gene is eliminated along with the vector segment (including the temperature sensitive replication origin and the drug resistance marker) from the chromosomal DNA by recombination of two of the glnE genes. In this case, the native glnE gene is left on the chromosomal DNA, and the deletion-type glnE gene is excised from the chromosomal DNA, or to the contrary, the deletion-type glnE gene is left on the chromosomal DNA, and the native glnE gene is excised from the chromosome DNA. In the both cases, the excised DNA is retained in the cell as a plasmid when the cell is cultured at a temperature at which the temperature sensitive replication origin can function. Subsequently, if the cell is cultured at a temperature at which the temperature sensitive replication origin cannot function, the glnE gene on the plasmid is eliminated along with the plasmid from the cell. Then, a strain in which glnE gene is disrupted can be obtained by selecting a strain in which the deletion-type glnE gene is left on the chromosome using PCR, Southern hybridization or the like.

Alternatively, gene disruption can be performed using a plasmid which cannot autonomously replicate in a coryneform bacterium in place of above-mentioned temperature sensitive plasmid. Examples of plasmids which cannot autonomously replicate in a coryneform bacterium include pHSG299 (Takara Shuzo) and pHSG399 (Takara Shuzo) and the like.

A strain having disrupted glnE can be obtained as described above.

The origin of the glnE gene used for the gene disruption is not particularly limited so long as the glnE gene has homology to the glnE gene originally contained in a coryneform bacterium to such a degree that homologous recombination occurs between them. For example, ATases of Mycobacterium tuberculosis (GenBank ACCESION Z70692) and ATase of Streptomyces coelicolor (GenBank ACCESSION Y17736) have homologies of 51.9% and 33.4% to the ATase of coryneform bacteria, respectively (Japanese Patent Laid-open No. 2002-300887, EP1229121).

(3) Reduction of PII Protein Activity

The inactivation of GS by adenylylation can also be affected by reducing the intracellular activity of PII protein. It is known that the PII protein is involved in the adenylylation of GS by ATase. The PII protein is a signal transfer protein for controlling the GS activity, and it is known to be uridylylated by uridylyl transferase (UTase). The uridylylated PII protein promotes deadenylylation of GS by ATase, and the deuridylylated PII protein promotes the adenylylation of GS by ATase.

It has been reported that GS is highly adenylylated in an UTase deficient strain (J. Bacteriology, 569-577, (134) 1978). This phenotype of excessive adenylylation is suppressed by mutation of the PII protein (J. Bacteriology, 816-822, (164) 1985). That is, the inactivation of GS by adenylylation can also be affected by reduction of PII protein activity. The reduction of PII protein activity means reduction of the function for promoting the adenylylation by ATase.

The reduction of the PII protein activity means, in addition to complete elimination of the activity, such reduction of the activity of the PII protein of the coryneform bacterium is lower than that of a wild-type strain or unmodified strain of the coryneform bacterium. For example, those in which the number of PII protein molecules per cell is reduced, those in which the specific activity of the PII protein per PII protein molecule is reduced, and so forth are encompassed Examples of the wild-type coryneform bacterium that serves as an object for comparison include, for example, the Brevibacterium lactofermentum ATCC 13869. The glnB gene encoding the PII protein of coryneform bacteria has been isolated, and it is suggested that the suppression of GS by the adenylylation of GS is affected by deletion of the gene (FEMS Microbiology Letters, 303-310, (173) 1999).

The gene glnb of Brevibacterium lactofermentum has already been described as a gene encoding the P11 protein, (EP1229121). The nucleotide sequence of the gene and the amino acid encoded thereby are shown in SEQ ID NO: 23 and SEQ ID NO: 24, respectively.

In the present invention, the PII protein, which is the object of the activity reduction, may have an amino acid sequence including deletion, substitution, insertion or addition of one or more amino acid residues in the aforementioned amino acid sequence of the PII protein at one or more sites.

The number of “one or more” amino acid residues varies depending on the position of the amino acid residues in the three-dimensional structure of the protein and type of amino acids. This is for the following reason. That is, some of amino acids are highly homologous to one another, and the difference between such amino acids does not significantly influence the three-dimensional structure of the protein. Thus, the mutant the PII protein of the present invention may be a protein exhibiting 30 to 50% or more, preferably 50 to 70% or more, more preferably 80 to 90% or more, most preferably 95% or more, homology with respect to the whole amino acid residues constituting the PII protein and having the PII protein activity.

The DNA encoding a protein substantially identical to the aforementioned PII protein includes a DNA that is hybridizable with the glnB gene under a stringent condition and codes for a protein having the PII protein activity. The aforementioned “stringent conditions” are conditions under which a so-called specific hybrid is formed, and a non-specific hybrid is not formed. For example, stringent conditions include a condition under which DNAs having high homology, for example, DNAs having homology of 50% or more, preferably 70% or more, more preferably 80% or more, most preferably 90% or more hybridize with each other, but DNAs having homology lower than the above do not hybridize with each other. Alternatively, stringent conditions include conditions whereby DNAs hybridize with each other at a salt concentrations corresponding to typical washing condition of Southern hybridization, i.e., approximately 1×SSC, 0.1% SDS, preferably 0.1×SSC, 0.1% SDS, at 60° C.

Examples of the method for reducing the PII protein activity of coryneform bacteria include, for example, a method of treating a coryneform bacterium with ultraviolet ray irradiation or a mutagenesis agent used in a typical mutagenesis treatment such as N-methyl-N′-nitro-N-nitrosoguanidine (NTG) or nitrous acid and selecting a mutant strain showing reduced activity of the PII protein. Besides the mutation treatment, a coryneform bacterium having reduced activity of the PII protein can also be obtained by transforming a coryneform bacterium with a DNA containing the glnB gene encoding the PII protein modified so as not to produce a PII protein that functions normally (deletion-type glnB gene) by deleting a part of the glnB gene to cause recombination between the deletion-type glnB gene and the glnB gene on a chromosome and thereby disrupt the glnB gene on a chromosome.

Gene destruction for the glnB gene can be carried out in the same manner as that for the aforementioned gene disruption of the glnE gene.

(4) Modification of Nitrogen Metabolism Mechanism

Adenylylation of GS may also be reduced or eliminated when a nitrogen metabolism regulation protein does not function normally.

The “nitrogen metabolism regulation protein” is a factor involved in the mechanism of changing GS into an inactive type by adenylylation of the tyrosine residue in the amino acid sequence of GS described above (nitrogen metabolism regulation mechanism comprising the adenylylation of glutamine synthetase as a major part), and includes a positive factor and a negative factor. The positive factor is a factor that increases the intracellular GS activity, and the negative factor is a factor that reduces the intracellular GS activity. The nitrogen metabolism regulation protein regulates not only GS but also an ammonium ion incorporation gene (ant, amtB). With increase of extracellular ammonium ion concentration, the nitrogen metabolism regulation protein reduces the activity of the incorporation gene such as amt and amtB, and thereby the incorporation of ammonium ions is suppressed.

It is known that, in Escherichia coli, when the intracellular glutamine concentration decreases, NR1, which is the positive factor of the nitrogen metabolism regulation protein, binds to the promoter regulating expression of the glnD gene, which codes for a uridylyl transferase (Utase). This occurs to increase the expression amount of glnD, and increase of uridylylated PII protein promotes the deadenylylation by ATase to increase the GS activity (Mol. Microbiology, (1998) (2) 29, 431-447).

It is known that, in Bacillus subtilis, when the intracellular glutamine concentration decreases, TnrA or GlnR, which are the negative factors of the nitrogen metabolism regulation protein, dissociates from the promoter that regulates the expression of the glnD gene encoding a uridylyl transferase (Utase). This occurs to increase the expression amount of glnD, and increase of uridylylated PII protein promotes the deadenylylation by ATase to increase the GS activity.

The nitrogen metabolism control mechanism utilizing the adenylylation of GS as a major mechanism is modified by the modification of the nitrogen metabolism regulation protein, and thereby the GS activity can be constantly increased. When the nitrogen metabolism regulator gene is a positive factor, the GS activity can be constantly increased by enhancing the activity of the factor, and when it is a negative factor, the GS activity can be constantly increased by reducing the activity of the factor.

In coryneform bacteria, the nitrogen metabolism control mechanism comprising the adenylylation of GS as a major part is controlled by the amtR gene product (AmtR), which is a negative nitrogen metabolism regulation protein (Mol. Microbiol., 37(4): 964-77, August 2000). Therefore, the GS activity can be increased by modifying the amtR gene so that AmtR does not function normally. The expression “AmtR does not function normally” means that the activity of AmtR is eliminated or reduced compared with a wild-type strain or unmodified strain of coryneform bacterium, and as a result, the GS activity is increased.

The nucleotide sequence of amtR and the amino acid encoded thereby of Brevibacterium lactofermentum are shown in SEQ ID NO: 21, and the amino acid sequence is shown in SEQ ID NO: 22.

In the present invention, AmtR, which is the object of the activity reduction, may have an amino acid sequence including deletion, substitution, insertion or addition of one or more amino acid residues in the aforementioned amino acid sequence of AmtR at one or more sites.

The number of “one or more” amino acid residues varies depending on the position in the three-dimensional structure of the protein and type of amino acid residue. Hoewver, the mutant AmtR of the present invention may be a protein exhibiting 30 to 50% or more, preferably 50 to 70% or more, more preferably 80 to 90% or more, most preferably 95% or more, of homology with respect to the whole amino acid residues constituting AmtR and having the AmtR activity.

The DNA encoding a protein substantially identical to the aforementioned AmtR includes a DNA that is hybridizable with the amtR gene under a stringent condition and codes for a protein having the AmtR activity. The aforementioned “stringent conditions”are conditions under which a so-called specific hybrid is formed, and a non-specific hybrid is not formed. For example, stringent conditions include conditions under which DNAs having high homology, for example, DNAs having homology of 50% or more, preferably 70% or more, more preferably 80% or more, most preferably 90% or more hybridize with each other, but DNAs having homology lower than the above does not hybridize with each other. Alternatively, stringent conditions include conditions whereby DNAs hybridize with each other at salt concentrations corresponding to typical washing conditions of Southern hybridization, i.e., approximately 1×SSC, 0.1% SDS, preferably 0.1×SSC, 0.1% SDS, at 60° C.

Examples of the method for modifying a coryneform bacterium so that AmtR is not function normally include, for example, treating a coryneform bacterium with ultraviolet ray irradiation or a mutagenesis agent used in a usual mutagenesis treatment such as N-methyl-N′-nitro-N-nitrosoguanidine (NTG) or nitrous acid and selecting a mutant strain showing reduced activity of AmtR. Besides the mutation treatment, a coryneform bacterium showing reduced activity of AmtR can also be obtained by transforming a coryneform bacterium with a DNA containing the amtR gene encoding AmtR modified so as not to produce AmtR that normally functions (deletion-type amtR gene) by deleting a part of the amtR gene to cause recombination between the deletion-type amtR gene and the amtR gene on a chromosome and thereby disrupt the amtR gene on a chromosome.

Such gene destruction for the amtR gene can be carried out in the same manner as that for the aforementioned gene disruption of the glnE gene.

The reduction or elimination of the adenylylation of GS may be attained by a combination of two or more of means selected from the mutation of GS such that GS is not be adenylylated, reduction of ATase activity, reduction of PII protein activity and modification of the nitrogen metabolism regulation protein described above.

Production of L-Arginine or L-Lysine Using Microorganism of the Present Invention

L-arginine or L-lysine can be efficiently produced by culturing a coryneform bacterium obtained as described above in a medium to produce and cause accumulation of L-arginine or L-lysine in the medium, and collecting the L-arginine or L-lysine from the medium.

Culture can be performed in a conventional manner using a typical medium containing a carbon source, nitrogen source and mineral salts as well as organic trace nutrients such as amino acids and vitamins, as required. Either a synthetic medium or a natural medium may be used. Any kind of carbon source and nitrogen source may be used so long as they can be utilized by a strain to be cultured.

As the carbon source, sugars such as glucose, glycerol, fructose, sucrose, maltose, mannose, galactose, starch hydrolysate and molasses, and organic acids such as acetic acid and citric acid can be used. In addition, alcohols such as ethanol can also be used each alone or in a combination with other carbon sources.

As the organic trace nutrients, amino acids, vitamins, fatty acids, nucleic acids, those containing those substances such as peptone, casamino acid, yeast extract and soybean protein decomposition product and so forth can be used. When an auxotrophic mutant that requires an amino acid or the like for its growth is used, it is preferable to supplement the required nutrient.

As the mineral salts, phosphates, magnesium salts, calcium salts, iron salts, manganese salts and so forth can be used.

The culture is preferably performed under aerobic conditions. The culture temperature is controlled to be between 20 to 45° C., and pH to be between 5 to 9. When pH falls during the culture, the medium is neutralized by addition of calcium carbonate or with an alkali such as ammonia gas or the like. A substantial amount of L-arginine or L-lysine is accumulated in the culture broth after 10 hours to 120 hours of culture in such a manner as described above.

After completion of the culture, L-arginine or L-lysine can be collected from the fermentation broth by a typical method. For example, after the cells were removed from the culture broth, L-arginine or L-lysine can be collected by concentrating the broth to crystallize L-glutamine.

### EXAMPLES

Hereinafter, the present invention will be explained more specifically with reference to the following non-limiting examples.

### Reference Example 1

### Construction of Coryneform Bacterium Deficient in Arginine Repressor

<1> Construction of Plasmid for argR Disruption

PCR was performed using chromosome DNA of a Brevibacterium flavum wild strain, 2247 strain (AJ14067) as a template and the oligonucleotides having the nucleotide sequences shown in SEQ ID NO: 1 (corresponds to the nucleotide numbers 4-28 in SEQ ID NO: 15) and SEQ ID NO: 2 (complementary to the sequence of the nucleotide numbers 4235-4211 in SEQ ID NO: 15) as primers. PCR was performed for 30 cycles each consisting of reactions at 98° C. for 10 seconds, 58° C. for 1 minute and 72° C. for 3 minutes using Pyrobest DNA polymerase (Takara Shuzo). The amplified fragment was inserted into the SmaI site in the multi-cloning site of cloning vector pHSG399.

In order to delete the whole ORF considered to encode the arginine repressor from the inserted DNA fragment, PCR was performed using the oligonucleotides having the nucleotide sequences shown in SEQ ID NO: 3 (corresponds to the nucleotide numbers 2372-2395 in SEQ ID NO: 15) and SEQ ID NO: 4 (complementary to the sequence of the nucleotide numbers 1851-1827 in SEQ ID NO: 15) as primers and pHSG399 inserted with the amplified fragment as a template. The PCR product was self-ligated to obtain pssER.

Then, a fragment, obtained by digesting pssER with restriction enzymes SmaI and SalI, and the temperature-sensitive plasmid pSFKT2 (see U.S. Pat. No. 6,303,383) digested with SmaI and SalI, were ligated to obtain a plasmid pssERT for argR disruption whose autonomous replication ability in coryneform bacteria became temperature-sensitive.

<2> Acquisition of Arginine Repressor Deficient Strain of Coryneform Bacterium by Homologous Recombination

The plasmid pssERT obtained as described above was introduced into a Brevibacterium lactofermentum wild-type strain, 2256 (ATCC13869). The plasmid was introduced by the electric pulse method (Japanese Patent Laid-open No. 2-207791). Because this plasmid showed temperature-sensitive autonomous replication ability in Brevibacterium lactofermentum, only strains in which this plasmid was incorporated into the chromosome by homologous recombination could be selected as kanamycin-resistant strains at 34° C., which was a temperature that did not allow for replication of the plasmid. Strains in which the plasmid for argR disruption was incorporated into a chromosome were selected as kanamycin-resistant strains on a CM2G plate (containing 10 g/L of polypeptone, 10 g/L of yeast extract, 5 g/L of glucose, 5 g/L of NaCl and 15 g/L of agar in 1 L of water, pH 7.2) containing 25 μg/ml of kanamycin. At this stage, native argR gene derived from the chromosome and the argR gene derived from the plasmid in which ORF was deleted were present in tandem on both sides of the plasmid portion on the chromosome.

Then, the recombinant strains were allowed to cause homologous recombination again, and strains that became kanamycin-sensitive were selected at 34° C., which was a temperature that did not allow for the plasmid replication to obtain strains in which one of the argR genes was eliminated. These strains included strains in which the native argR gene remained on the chromosome and strains in which the disrupted argR gene remained on the chromosome. From these strains, a strain having only the disrupted argR gene was selected. An argR gene on the chromosome could be determined to be the disrupted-type by preparing chromosome of a strain that became kanamycin-sensitive at 34° C., performing PCR utilizing the chromosome as a template and the oligonucleotides having the nucleotide sequences shown in SEQ ID NOS: 1 and 2 as primers, and confirming that the PCR product was shorter by about 600 bp than that obtained by similarly performing PCR utilizing chromosome derived from the parent strain as a template.

Direct sequencing of the PCR product of the argR-disrupted strain selected as described above was performed to confirm that the argR gene was disrupted as desired, and thus 2256DR stain was obtained. This strain produces a markedly larger amount of L-arginine compared with the parent strain (see US2002045223 A1).

### Example 1

### Construction of Adenylyltransferase (GlNE) Deficient Strain

<1> Preparation of Plasmid for Deletion of GlnE

The glnE sequence from Brevibacterium flavum ATCC 14067 has been reported (EP 1229121A2). Based on the reported nucleotide sequence, the primers shown in SEQ ID NOS: 5 and 6 were synthesized and used together with the chromosomal DNA of Brevibacterium lactofermentum ATCC 13869 strain as a template to amplify an internal sequence of glnE by the PCR method.

The chromosomal DNA of Brevibacterium lactofermentum ATCC 13869 strain was prepared by using Bacterial Genome DNA Purification Kit (Advanced Genetic Technologies Corp.). PCR was performed for 30 cycles each consisting of denaturation at 94° C. for 30 seconds, annealing at 55° C. for 15 seconds and extension at 72° C. for 2 minutes using Pyrobest DNA Polymerase (Takara Shuzo).

The PCR product was purified in a conventional manner and blunt-ended using Blunting Kit (Takara Shuzo). The blunt-ended PCR product was ligated to HincII site of pHSG299 using Ligation Kit (Takara Shuzo), and used to transform competent cells of Escherichia coli JM109 (Takara Shuzo). The cells were plated on L medium containing 10 μg/ml of IPTG, 40 μg/ml of X-Gal and 25 μg/ml of kanamycin and cultured overnight. Then, the emerged white colonies were picked up and separated into single colonies to obtain transformants.

Plasmids are prepared from the transformants by the alkali method, and structures of the plasmids were confirmed. A plasmid in which a partial fragment of glnE was inserted into the vector was designated as pAATase-299.

The aforementioned pAATase-299 does not include any sequence autonomously replicable in coryneform bacteria. Therefore, if a coryneform bacterium is transformed with this plasmid, a strain in which this plasmid is incorporated into a chromosome by homologous recombination appears as a transformant, although it occurs at an extremely low frequency.

<2> Introduction of pΔATase-299 into Arginine Repressor-Deficient Strain and Evaluation of Amino Acid Production

The arginine repressor-deficient strain obtained in Reference Example 1, 2256ΔargR strain, was transformed with the plasmid pΔATase-299 by the electric pulse method (see Japanese Patent Laid-open No. 2-207791), and transformants were obtained by using the kanamycin-resistance as a marker. A sequence around the glnE gene of each transformant was amplified by PCR, and disruption of the glnE gene was confirmed to obtain a strain in which glnE of 2256ΔargR was disrupted (2256ΔargRΔglnE). Using the obtained transformant 2256ΔargRΔglnE, culture for production of L-arginine and L-lysine was performed as follows.

Cells of 2256ΔargRΔglnE obtained by culture on a CM2G plate medium containing 25 μg/ml of kanamycin were inoculated into a medium containing 40 g of glucose, 65 g of H₄)₂SO₄, 1 g of KH₂PO₄, 0.4 g of MgSO₄0.7H₂O, 0.01 g of FeSO₄, 0.01 g of MnSO₄, 50 μg of VB₁-HCl, 50 μg of biotin, 45 mg (amount of N) of soybean hydrolysates and 50 g of CaCO₃ in 1 L of pure water (adjusted to pH 7.0 with KOH) and cultured at 31.5° C. with shaking until the sugar in the medium was consumed.

After completion of the culture, the amount of accumulated L-arginine (Arg) in the culture broth was analyzed by liquid chromatography for appropriately diluted culture broth. The accumulated L-lysine amount (Lys) and accumulated L-glutamic acid amount (Glu) were analyzed for appropriately diluted culture broth by using Biotech Analyzer (Asahi Chemical Industry). The accumulated L-glutamine amount (Gln) and accumulated N-acetylglutamic acid amount was analyzed by liquid chromatography for appropriately diluted culture broth. The results are shown in Table 1.

The GS activity of each of the aforementioned strains was also measured. The GS activity was measured by adding a crude enzyme solution to a solution containing 100 mM imidazole-HCl (pH 7.0), 0.1 mM NH₄Cl, 1 mM MnCl₂, 1 mM phosphoenolpyruvic acid, 0.3 mM NADH, 10 U of lactate dehydrogenase, 25 U of pyruvate kinase, 1 mM ATP and 10 mM monosodium glutamate (MSG) and measuring variation of absorbance at 340 nm at 30° C. referring to the method described in Journal of Fermentation and Bioengineering, Vol. 70, No. 3, 182-184, 1990. For the measurement of blank, the aforementioned reaction solution not containing MSG was used. The crude enzyme solution was prepared by separating cells from the aforementioned culture broth by centrifugation, washing the cells with 100 mM imidazole-HCl (pH 7.0), sonicating the cells and removing undisrupted cells by centrifugation. The protein concentration of the crude enzyme solution was quantified by using Protein Assay (Bio-Rad) with bovine serum albumin as a standard sample. The results are shown in Table 2.

TABLE 1

| | Arg, | Lys. | Glu, | Gln, | N-Acetylglutamic |
|---|---|---|---|---|---|
| Strain | (g/L) | (g/L) | (g/L) | (g/L) | Acid, (g/L) |
| 2256ΔargR | 2.28 | 0.81 | 0.32 | 0.19 | 0.0153 |
| 2256ΔargRΔglnE | 3.18 | 1.32 | 0.4 | 0.2 | 0.0381 |

TABLE 2

| | GS activity |
|---|---|
| Strain | (nmol/min/mg) |
| 2256ΔargR | 51.2 |
| 2256ΔargRΔglnE | 123.1 |

In the GlnE-deficient strain, improvement of accumulation of L-arginine and L-lysine was observed compared with the parent strain, 2256ΔargR. The specific activity of GS was improved in the GlnE-deficient strain by about 2.4 times compared with the parent strain. Moreover, increase of N-acetylglutamic acid and glutamic acid, which are the precursors of L-arginine, was also observed.

The 2256ΔargRΔglnE strain was given with a private numver AJ110145, and it was deposited at the independent administrative agency, National Institute of Advanced Industrial Science and Technology, International Patent Organism Depository (postal code 305-5466, Tsukuba Central 6, 1-1 Higashi 1-Chome, Tsukuba-shi, Ibaraki-ken, Japan) on Feb. 18, 2003 and given with an accession number of FERM P-19216. Then, it was converted to an international deposit based on the Budapest Treaty on Feb. 19, 2004 and given an accession number of FERM BP-08630.

### Example 2

### Evaluation of GS Adenylylation Site-Modified Strain

<1> Construction of GlNA Adenylylation Site-Modified Plasmid

The adenylylation site of glnA gene product (GlnA) of coryneform bacteria has been reported (FEMS Microbiology Letters, 303-310, (173)1999). Therefore, GlnA adenylylation site-modified strain was obtained by replacing the glnA gene on the chromosome with a glnA gene having a modified adenylylation site of GINA. Specific procedures will be described herein.

First, PCR was performed using chromosome DNA of Brevibacterium lactovermentum ATCC13869 strain as a template and the synthetic DNAs shown in SEQ ID NOS: 7 and 8 as primers to obtain an amplification product for the N-terminus side of the glnA gene. Separately, to obtain an amplification product for the C-terminus side of the glnA gene, PCR was performed using chromosome DNA of Brevibacterium lactofermentum ATCC 13869 strain as a template and the synthetic DNAs shown in SEQ ID NOS: 9 and 10 as primers. Since mismatches were introduced into the sequences shown in SEQ ID NOS: 8 and 9, a mutation was introduced into the terminal portion of each of the amplification products. Specifically, the tyrosine residue at position 405 of GlnA is replaced with an L-phenylalanine residue by this mutation.

Then, in order to obtain a glnA gene fragment introduced with a mutation, PCR was performed by using the aforementioned gene products for N- and C-terminus sides of glnA mixed in equimolar amounts as a template and the synthetic DNAs shown in SEQ ID NOS: 10 and 11 as primers to obtain a glnA gene amplification product introduced with a mutation at the adenylylation site. The PCR product was purified in a conventional manner, digested with HincII and inserted into the HincII site of pHSG299 (Takara Shuzo). This plasmid was designated as pGSA2.

Since the above pGSA2 does not contain a region that enables its autonomous replication in coryneform bacteria, when a coryneform bacterium is transformed with this plasmid, a strain in which the plasmid is incorporated into chromosome by homologous recombination is obtained as a transformant, although it occurs at an extremely low frequency.

<2> Introduction of pGSA2 into Arginine Repressor-Deficient Strain and Evaluation of Amino Acid Production

The ArgR-deficient strain derived from Brevibacterium lactofermentum ATCC 13869 described in Reference Example 1, 2256ΔR, was transformed with the plasmid pGSA2 of a high concentration by the electric pulse method (see Japanese Patent Laid-open No. 2-207791), and transformants were obtained by using the kanamycin-resistance as a marker. The glnE gene sequence of each transformant was determined, and a transformant in which the adenylylation site in the sequence was replaced with that region in glnA derived from pGSA2 was designated 2256ΔargRAde. By using the 2256ΔargR strain and 2256ΔargRAde strain, culture for L-arginine and L-lysine production and measurement of GS enzymatic activity were performed in the same manner as that described in Example 1, <2>. The results are shown in Tables 3 and 4.

TABLE 3

| | Arg | Lys | Glu | Gln | N-Acetylglutamic |
|---|---|---|---|---|---|
| Strain | (g/L) | (g/L) | (g/L) | (g/L) | acid, (g/L) |
| 2256ΔargR | 2.28 | 0.81 | 0.32 | 0.19 | 0.0153 |
| 2256ΔargRAde | 2.88 | 1.48 | 0.49 | 0.31 | 0.314 |

TABLE 4

| | GS activity |
|---|---|
| Strain | (nzmol/min/mg) |
| 2256ΔargR | 51.2 |
| 2256ΔargRAde | 135.1 |

In the 2256ΔargRAde strain, improvement of L-arginine and L-lysine accumulations was observed as compared with 2256ΔargR. Moreover, increase of L-glutamic acid, L-glutamine and N-acetylglutamic acid, which are the precursors of L-arginine, was also observed. The specific activity of GS increased by about 2.6 times.

The 2256ΔargRAde strain was given a private number of AJ110146, and it was deposited at the independent administrative agency, National Institute of Advanced Industrial Science and Technology, International Patent Organism Depository (postal code 305-5466, Tsukuba Central 6, 1-1 Higashi 1-Chome, Tsukuba-shi, Ibaraki-ken, Japan) on Feb. 18, 2003 and given with an accession number of FERM P-19217. Then, it was converted to an international deposit based on the Budapest Treaty on Feb. 19, 2004 and given an accession number of FERM BP-08631.

### Example 3

### Acquisition and Evaluation of AmtR-Deficient Strain

<1> Preparation of Plasmid for Deletion of AmtR

A plasmid for deleting the amtR gene product (AmtR) of coryneform bacteria was prepared as follows.

First, a chromosomal DNA of Brevibacterium lactofermentum ATCC 13869 strain was extracted and used as a template together with the synthetic DNAs shown in SEQ ID NOS: 13 and 14 as primers to perform PCR. The obtained DNA fragment was blunt-ended and inserted into pHSG299 (Takara Shuzo) at the HincII site. The obtained plasmid was designated pΔamtRT.

The aforementioned pΔamtR does not include any sequence enabling autonomous replication within a cell of coryneform bacteria. Therefore, if a coryneform bacterium is transformed with this plasmid, a strain in which this plasmid is incorporated into a chromosome by homologous recombination appears as a transformant, although it occurs at an extremely low frequency.

<2> Introduction of pΔamtR into Arginine Repressor-Deficient Strain and Evaluation of Culture

The arginine repressor-deficient strain obtained in Reference Example 1, 2256ΔargR strain, was transformed with pΔamtR, and transformants were obtained by using the kanamycin-resistance as a marker. A sequence around the amtR gene of each transformant was determined, and disruption of the amtR gene was confirmed to obtain an amtR-disrupted strain of 2256ΔargR (2256ΔargRΔamtR). Using the obtained transformant 2256ΔargRΔamtR, culture for L-arginine and L-lysine production was performed in the same manner as that described in Example 1, <2>. The results are shown in Tables 5 and 6.

TABLE 5

| | Arg, | Lys, | Glu, | Gln, | N-Acetylglutamic |
|---|---|---|---|---|---|
| Strain | (g/L) | (g/L) | (g/L) | (g/L) | acid, (g/L) |
| 2256ΔargR | 2.28 | 0.81 | 0.32 | 0.19 | 0.0153 |
| 2256ΔargRΔamtR | 3.16 | 1.48 | 0.45 | 0.41 | 0.0296 |

TABLE 6

| | GS activity |
|---|---|
| Strain | (nmol/min/mg) |
| 2256ΔargR | 51.2 |
| 2256ΔargRΔamtR | 132.3 |

In the 2256ΔargRΔamtR strain, improvement of L-arginine and L-lysine accumulations was observed compared with 2256ΔargR. Moreover, accumulation of glutamic acid, N-acetylglutamic acid and glutamine, which are the precursors of L-arginine, was also observed. The specific activity of GS increased by about 2.6 times.

The 2256ΔargRΔamtR strain was given a private number of AJ110144, and it was deposited at the independent administrative agency, National Institute of Advanced Industrial Science and Technology, International Patent Organism Depository (postal code 305-5466, Tsukuba Central 6, 1-1 Higashi 1-Chome, Tsukuba-shi, Ibaraki-ken, Japan) on Feb. 18, 2003 and given with an accession number of FERM P-19215. Then, it was converted to an international deposit based on the Budapest Treaty on Feb. 19, 2004 and given an accession number of FERM BP-08629.

While the invention has been described in detail with reference to preferred embodiments thereof, it will be apparent to one skilled in the art that various changes can be made, and equivalents employed, without departing from the scope of the invention. Each of the aforementioned documents, including the foreign priority document, JP 2003-56129, is incorporated by reference herein in its entirety.

[Explanation of Sequence Listing]

SEQ ID NO: 1: Primer for argR gene amplification
SEQ ID NO: 2: Primer for argR gene amplification
SEQ ID NO: 3: Primer for amplification of a part of the plasmid containing the argR gene other than the argR gene ORF
SEQ ID NO: 4: Primer for amplification of a part of the plasmid containing the argR gene other than the argR gene ORF
SEQ ID NO: 5: Primer N for glnE disruption
SEQ ID NO: 6: Primer C for glnE disruption
SEQ ID NO: 7: GlNA adenylylation 1 st PCR primer NN
SEQ ID NO: 8: GlNA adenylylation 1 st PCR primer NC
SEQ ID NO: 9: GlNA adenylylation 1 st PCR primer CN
SEQ ID NO: 10: GlnA adenylylation 1st PCR primer CC
SEQ ID NO: 11: glnA 2nd PCR primer N
SEQ ID NO: 12: glnA 2nd PCR primer C
SEQ ID NO: 13: Primer N for amtR disruption
SEQ ID NO: 14: Primer C for amtR disruption
SEQ ID NO: 15: nucleotide sequence of argR gene
SEQ ID NO: 16: amino acid sequence encoded by the above DNA fragment
SEQ ID NO: 17: nucleotide sequence of glnE gene
SEQ ID NO: 18: amino acid sequence encoded by the above DNA fragment
SEQ ID NO: 19: nucleotide sequence of glnA gene
SEQ ID NO: 20: amino acid sequence encoded by the above DNA fragment
SEQ ID NO: 21: nucleotide sequence of amtR gene
SEQ ID NO: 22: aino acid sequence encoded by the above DNA fragment
SEQ ID NO: 23: ncleotide sequence of glnB gene
SEQ ID NO: 24: amino acid sequence encoded by the above DNA fragment

1. A coryneform bacterium which has an L-arginine- or L-lysine-producing ability, and wherein said bacterium is modified so that glutamine synthetase activity is enhanced as compared to a wild-type coryneform bacterium.

2. The coryneform bacterium of claim 1, which comprises a modification that results in adenylylation of glutamine synthetase being reduced or eliminated.

3. The coryneform bacterium of claim 2, wherein said modification is selected from the group consisting of
a) mutating the adenylylation site of glutamine synthetase;
b) reducing the intracellular activity of glutamine synthetase adenylyltransferase,
c) reducing the intracellular activity of PII protein, and
d) increasing the intracellular activity of glutamine synthetase by modifying a nitrogen metabolism regulation protein.

4. The coryneform bacterium of claim 3, wherein said adenylylation site comprises position 405 of SEQ ID NO: 20 and wherein said mutation comprises replacement of the wild-type tyrosine residue with another amino acid.

5. The coryneform bacterium of claim 3, wherein a gene encoding the glutamine synthetase adenylyltransferase on a chromosome of said bacterium is disrupted.

6. The coryneform bacterium of claim 3, wherein the nitrogen metabolism regulation protein is an amtR gene product which does not function normally,

7. The coryneform bacterium of claim 6, wherein said amtR gene product on a chromosome of said bacterium is disrupted.

8. The coryneform bacterium according to claim 1, which is modified so that an arginine repressor does not function normally.

9. The coryneform bacterium of claim 8, wherein a gene on a chromosome of said bacterium encoding the arginine repressor is disrupted.

10. A method for producing L-arginine or L-lysine, comprising the steps of
a) culturing the coryneform bacterium according to claim 1 in a medium, and
b) allowing accumulation of L-arginine or L-lysine in the medium, and
c) collecting the L-arginine or L-lysine from the medium.


SEQUENCE LISTING (24 sequences)

SEQ ID NO: 1 - DNA, 25 bp, Artificial Sequence
  Description of Artificial Sequenceprimer for PCR
cccgggttttcttctgcaactcggg

SEQ ID NO: 2 - DNA, 25 bp, Artificial Sequence
  Description of Artificial Sequenceprimer for PCR
gtcgacaagctcggttgttcccagc

SEQ ID NO: 3 - DNA, 24 bp, Artificial Sequence
  Description of Artificial Sequenceprimer for PCR
cccctagttcaaggcttgttaatc

SEQ ID NO: 4 - DNA, 25 bp, Artificial Sequence
  Description of Artificial Sequenceprimer for PCR
gtcttacctcggctggttggccagc

SEQ ID NO: 5 - DNA, 23 bp, Artificial Sequence
  Description of Artificial Sequence primer
agaactacgagtccgcctttttg

SEQ ID NO: 6 - DNA, 21 bp, Artificial Sequence
  Description of Artificial Sequence primer
cgatcaccagcaacccacgca

SEQ ID NO: 7 - DNA, 22 bp, Artificial Sequence
  Description of Artificial Sequence primer
cttcccagtagcaccatacgac

SEQ ID NO: 8 - DNA, 26 bp, Artificial Sequence
  Description of Artificial Sequence primer
ctggtggcagttcgaagaggtccttg

SEQ ID NO: 9 - DNA, 26 bp, Artificial Sequence
  Description of Artificial Sequence primer
ggacaaggacctcttcgaactgccag

SEQ ID NO: 10 - DNA, 26 bp, Artificial Sequence
  Description of Artificial Sequence primer
cggcgagaccgtcgattgggaggagc

SEQ ID NO: 11 - DNA, 22 bp, Artificial Sequence
  Description of Artificial Sequence primer
gtagcaccttacgaccaaaccg

SEQ ID NO: 12 - DNA, 20 bp, Artificial Sequence
  Description of Artificial Sequence primer
ggagccggtcgacgaggagc

SEQ ID NO: 13 - DNA, 23 bp, Artificial Sequence
  Description of Artificial Sequence primer
gccccgggcaggcaagaatcctc

SEQ ID NO: 14 - DNA, 21 bp, Artificial Sequence
  Description of Artificial Sequence primer
tccccgggaggctctctgcgg

SEQ ID NO: 15 - DNA, 4235 bp, Brevibacterium flavum
  CDS (1852)..(2364)
aaacccgggttttcttctgcaactcgggcgccgaagcaaacgaggctgctttcaagattgcacgcttgactggtcgttcccggattctggctgcagttcatggtttccacggccgcaccatgggttccctcgcgctgactggccagccagacaagcgtgaagcgttcctgccaatgccaagcggtgtggagttctacccttacggcgacaccgattacttgcgcaaaatggtagaaaccaacccaacggatgtggctgctatcttcctcgagccaatccagggtgaaacgggcgttgttccagcacctgaaggattcctcaaggcagtgcgcgagctgtgcgatgagtacggcatcttgatgatcaccgatgaagtccagactggcgttggccgtaccggcgatttctttgcacatcagcacgatggcgttgttcccgatgtggtgaccatggccaagggacttggcggcggtcttcccatcggtgcttgtttggccactggccgtgcagctgaattgatgaccccaggcaagcacggcaccactttcggtggcaacccagttgcttgtgcagctgccaaggcagtgctgtctgttgtcgatgacgctttctgcgcagaagttacccgcaagggcgagctgttcaaggtacttcttgccaaggttgacggcgttgtagacgtccgtggcaggggcttgatgttgggcgtggtgctggagcgcgacgtcgcaaagcaagctgttcttgatggttttaagcacggcgttattttgaatgcaccggcggacaacattatccgtttgaccccgccgctggtgatcaccgacgaagaaatcgcagacgcagtcaaggctattgccgagacaatcgcataaaggacttaaacttatgacttcacaaccacaggttcgccatttcctggctgatgatgatctcacccctgcagagcaggcagaggttttgaccctagccgcaaagctcaaggcagcgccgttttcggagcgtccactcgagggaccaaagtccgttgcagttctttttgataagacttcaactcgtactcgcttctccttcgacgcgggcatcgctcatttgggtggacatgccatcgtcgtggattccggcagctcacagatgggtaagggcgagaccctgcaggacaccgcagctgtattgtcccgctacgtggaagcaattgtgtggcgcacctacgcacacagcaatttccacgccatggcggagacgtccactgtgccgctggtgaactccttgtccgatgatctgcacccatgccagattctggctgatctgcagaccatcgtggaaaacctcagccctgaagaaggcccagcaggccttaagggtaagaaggctgtgtacctgggcgatggcgacaacaacatggccaactcctacatgattggctttgccaccgcgggcatggatatttccatcatcgctcctgaagggttccagcctcgtgcggaattcgtggagcgcgcggaaaagcgtggccaggaaaccggcgcgaaggttgttgtcaccgacagcctcgacgaggttgccggcgccgatgttgtcatcaccgatacctgggtatccatgggtatggaaaacgacggcatcgatcgcaccacacctttcgttccttaccaggtcaacgatgaggtcatggcgaaagctaacgacggcgccatcttcctgcactgccttcctgcctaccgcggcaaagaagtggcagcctccgtgattgatggaccagcgtccaaagttttcgatgaagcagaaaaccgcctccacgctcagaaagcactgctggtgtggctgctggccaaccagccgaggtaagacatgtctcttggctcaaccccgtcaacaccggaaaacttaaatcccgtgactcgcactgcacgccaagctctcattttgcagattttggacaaacaaaaagtcaccagccaggtacaactgtctgaattgctgctggatgaaggcatcgatatcacccaggccaccttgtcccgggatctcgatgaactcggtgcacgcaaggttcgccccgatgggggacgcgcctactacgcggtcggcccagtagatagcatcgcccgcgaagatctccggggtccgtcggagaagctgcgccgcatgcttgatgaactgctggtttctacagatcattccggcaacatcgcgatgctgcgcaccccgccgggagctgcccagtacctggcaagtttcatcgatagggtggggctgaaagaagtcgttggcaccatcgctggcgatgacaccgtttttgttctcgcccgtgatccgctcacaggtaaagaactaggtgaattactcagcgggcgcaccacttaaagcgcccctagttcaaggcttgttaatcgcttgttaatgcaggcaggtaaggtataacccgagtgttttttcgaggaataccaaccctttcaacacaataattttctttaaacatccttgctgtccaccacggctggcaaggaacttaaaatgaaggagcacacctcatgactaaccgcatcgttcttgcatactccggcggtctggacaccactgtggcaattccatacctgaagaagatgattgatggtgaagtcatcgcagtttctctcgacctgggccagggtggagagaacatggacaacgttcgccagcgtgcattggatgccggtgcagctgagtccatcgttgttgatgcaaaggatgagttcgctgaggagtactgcctgccaaccatcaaggcaaacggcatgtacatgaagcagtacccactggtttctgcaatctcccgcccactgatcgtcaagcacctcgttgaggctggcaagcagttcaacggtacccacgttgcacacggctgcactggtaagggcaacgaccaggttcgtttcgaggtcggcttcatggacaccgatccaaacctggagatcattgcacctgctcgtgacttcgcatggacccgcgacaaggctatcgccttcgccgaggagaacaacgttccaatcgagcagtccgtgaagtccccattctccatcgaccagaacgtctggggccgcgctattgagaccggttacctggaagatctgtggaatgctccaaccaaggacatctacgcatacaccgaggatccagctctgggtaacgctccagatgaggtcatcatctccttcgagggtggcaagccagtctccatcgatggccgtccagtctccgtactgcaggctattgaagagctgaaccgtcgtgcaggcgcacagggcgttggccgccttgacatggttgaggaccgtctcgtgggcatcaagtcccgcgaaatctacgaagcaccaggcgcaatcgcactgattaaggctcacgaggctttggaagatgtcaccatcgagcgcgaactggctcgctacaagcgtggcgttgacgcacgttgggctgaggaagtatacgacggcctgtggttcggacctctgaagcgctccctggacgcgttcattgattccacccaggagcacgtcaccggcgatatccgcatggttctgcacgcaggttccatcaccatcaatggtcgtcgttccagccactccctgtacgacttcaacctggctacctacgacaccggcgacaccttcgaccagaccctggctaagggctttgtccagctgcacggtctgtcctccaagatcgctaacaagcgcgatcgcgaagctggcaacaactaagccaccttttcaagcatccagactagaacttcaagtatttagaaagtagaagaacaccacatggaacagcacggaaccaatgaaggtgcgctgtggggcggccgcttctccggtggaccctccgaggccatgttcgccttgagtgtctccactcatttcgactgggttttggccccttatgatgtgttggcctccaaggcacacgccaaggttttgcaccaagcagagctactttctgatgaagatctagccaccatgctggctggtcttgatcagctgggcaaggatgtcgccgacggaaccttcggtccgctgccttctgatgaggatgtgcacggcgcgatggaacgcggtctgattgaccgcgttggtcctgaggtgggcggccgtctgcgcgctggtcgttcccgcaacgaccaggtggcaaccctgttccgcatgtgggtccgcgacgcagtgcgcgacatcgcgctgggaacaaccgagcttgtcgac

SEQ ID NO: 16 - protein, 171 aa, Brevibacterium flavum
MSLGSTPSTPENLNPVTRTARQALILQILDKQKVTSQVQLSELLLDEGIDITQATLSRDLDELGARKVRPDGGRAYYAVGPVDSIAREDLRGPSEKLRRMLDELLVSTDHSGNIAMLRTPPGAAQYLASFIDRVGLKEVVGTIAGDDTVFVLARDPLTGKELGELLSGRTT

SEQ ID NO: 17 - DNA, 3138 bp, Brevibacterium lactofermentum
  CDS (1)..(3138)
atgtcaggaccgttaagaagtgaacgtaaagtcgttggctttgtcagagacccactgccaaaagttggttctttatcgctgaaatctgagcatgcccaagcagatctagagcatttgggttggcgcaatgttgagtctttggatttgttgtggggcttgtcaggtgcaggcgatcccgatgtcgcgctgaaccttcttattcggctgtatcaggcacttgaagcaatcggcgaggatgctcgaaacgagcttgatcaagagattcgccaggatgaagaactacgagtccgcctttttgcattgttgggtggttcctcggctgtcggtgatcacttggtcgccaatcctttgcagtggaaactcttaaaacttgatgcgccatcgagggaagagatgtttcaggcgctgctggaatctgtgaaagctcagcctgctgtgcttgaggttgaggatttcagcgatgcacacaacattgcccgagacgatttgagcacgcctggtttttacacggctagtgttaccgggcctgaagcagagcgagtcttgaaatggacttatcgcacgttgctgacccggattgctgcgcatgatttagcgggtacctatcccaccgacatgcggagaaaaggtggcgatcctgttccgtttagcacagtgaccatgcagctcagcgacctagctgatgctgctttgactgctgctttagctgtggcaattgccaatgtttatggtgaaaagccggttgattcagctttatctgtcatcgcgatgggcaaatgtggcgcgcaggaattgaactacatttcagatgtggacgtggtgtttgttgcagagccggcaaactctaaatcaacacgcaccgcagcagagctcattcgcatcggtagcaactcgttctttgaggtggatgcagcacttcgcccagaaggtaaaagtggcgctcttgtgcgctctttggattcccatatggcgtattacaagcgctgggcggaaacctgggaatttcaggcactgctgaaagctcgtcccatgacgggtgatattgaccttgggcagtcctatgtggatgctctttcaccgttgatttgggcggctagccagcgggaatcatttgtcacagatgtccaagctatgcgccgtcgagtgttggacaatgttccggaagacttgcgtgatcgtgagctgaagcttggtcgcggtggtttgagggatgtggagtttgctgtccagctccttcagatggtgcatggtcgcattgatgagacgttgcgggttcggtcaacggtaaatgctttgcatgtgttggttgatcagggatatgtgggtcgtgaagacgggcataatctcattgagtcgtatgagtttttgcgcctgttggagcatcgccttcaattggagcggatcaagcgcactcacttgttaccgaaacctgatgaccgaatgaatatgcgctggttggcgcgcgcttctgggtttactggttcgatggagcaaagttcggccaaagctatggaacggcatttgcgtaaggttcgtttgcagattcagtcgttgcatagtcagctgttttatcggccactgctgaactctgtggtcaacttgagcgcggatgccatcagattgtctccggatgctgcaaagctacaattgggggcattgggatacctgcatccatcacgtgcttatgaacacctgactgctcttgcatcaggagctagccgtaaagccaagattcaggcgatgttgctgcccacgttgatggagtggctgtctcaaacagctgaaccagatgcgggattgctgaattaccgcaagctttctgatgcttcctatgatcgcagctggtttttgcgcatgctgcgtgatgagggcgtagtggggcagcggttgatgcgtattttgggaaattctccctatatttctgaactgattatctccactccggactttgtgaaacagctgggtgatgcggcgtctggtcctaaattgcttgctactgcaccgactcaggttgtgaaagcaatcaaggcgacggtgtcgcgtcatgagtcacctgatcgggcgatccaggctgcacgatcgctgaggaggcaggagctggcacgcattgcctctgctgatttgctcaacatgctcactgttcaggaagtatgccaaagcttgtcactagtctgggatgcggtgttggatgctgccttggatgcggaaatccgtgctgcacttaacgatccacagaaaccagatcagcctctggccaatatttctgtgatcggcatgggccgtttgggtggagcagaacttggatacggttctgatgccgatgtgatgtttgtatgcgagccggtagccggtgtggaagagcatgaggccgtcacatggtctattgcgatctgtgattccatgcggtcgaggcttgcgcagccttccggtgatccacctttggaggtggatctggggctgcgtcctgaagggagatctggtgcgattgtgcgcaccgttgattcctatgtgaagtactacgaaaagtggggtgaaacttgggagattcaggcgctgctgagggctgcgtgggttgctggtgatcgtgagctgggcattaagttcttggagtcgattgatcgtttccgctacccagttgacggggcaacgcaggcgcagcttcgtgaagttcgtcgaattaaggcgagggtggataatgagaggcttccgcgcggggctgatcgaaatacccataccaagctgggtcggggagcgttaactgacatcgagtggactgtgcagttgttgaccatgatgcatgctcatgagattccggagctgcacaatacgtcgacgttggaagttcttgaagtgctggaaaagcatcagattattaaccctgtgcaggtgcagacgcttcgggaagcgtggctgacggcaacggctgctaggaatgcgcttgtgctggtcaggggtaagagattagatcagttacctactcctggtccgcaccttgcgcaggtggctggtgcgtctggttgggatccaaatgagtaccaggagtatttggaaaactatctgaaagtgaccaggaagagtcgtcaggttgttgatgaagtcttctggggtgtggactctatggagcaacgtgagttttag

SEQ ID NO: 18 - protein, 1045 aa, Brevibacterium lactofermentum
MSGPLRSERKVVGFVRDPLPKVGSLSLKSEHAQADLEHLGWRNVESLDLLWGLSGAGDPDVALNLLIRLYQALEAIGEDARNELDQEIRQDEELRVRLFALLGGSSAVGDHLVANPLQWKLLKLDAPSREEMFQALLESVKAQPAVLEVEDFSDAHNIARDDLSTPGFYTASVTGPEAERVLKWTYRTLLTRIAAHDLAGTYPTDMRRKGGDPVPFSTVTMQLSDLADAALTAALAVAIANVYGEKPVDSALSVIAMGKCGAQELNYISDVDVVFVAEPANSKSTRTAAELIRIGSNSFFEVDAALRPEGKSGALVRSLDSHMAYYKRWAETWEFQALLKARPMTGDIDLGQSYVDALSPLIWAASQRESFVTDVQAMRRRVLDNVPEDLRDRELKLGRGGLRDVEFAVQLLQMVHGRIDETLRVRSTVNALHVLVDQGYVGREDGHNLIESYEFLRLLEHRLQLERIKRTHLLPKPDDRMNMRWLARASGFTGSMEQSSAKAMERHLRKVRLQIQSLHSQLFYRPLLNSVVNLSADAIRLSPDAAKLQLGALGYLHPSRAYEHLTALASGASRKAKIQAMLLPTLMEWLSQTAEPDAGLLNYRKLSDASYDRSWFLRMLRDEGVVGQRLMRILGNSPYISELIISTPDFVKQLGDAASGPKLLATAPTQVVKAIKATVSRHESPDRAIQAARSLRRQELARIASADLLNMLTVQEVCQSLSLVWDAVLDAALDAEIRAALNDPQKPDQPLANISVIGMGRLGGAELGYGSDADVMFVCEPVAGVEEHEAVTWSIAICDSMRSRLAQPSGDPPLEVDLGLRPEGRSGAIVRTVDSYVKYYEKWGETWEIQALLRAAWVAGDRELGIKFLESIDRFRYPVDGATQAQLREVRRIKARVDNERLPRGADRNTHTKLGRGALTDIEWTVQLLTMMHAHEIPELHNTSTLEVLEVLEKHQIINPVQVQTLREAWLTATAARNALVLVRGKRLDQLPTPGPHLAQVAGASGWDPNEYQEYLENYLKVTRKSRQVVDEVFWGVDSMEQREF

SEQ ID NO: 19 - DNA, 1434 bp, Brevibacterium lactofermentum
  CDS (1)..(1434)
gtggcgtttgaaaccccggaagaaattgtcaagttcatcaaggatgaaaacgtcgagttcgttgacgttcgattcaccgaccttcccggcaccgagcagcacttcagcatcccagctgccagcttcgatgcagatacagtcgaagaaggtctcgcattcgacggatcctcgatccgtggcttcaccacgatcgacgaatctgacatgaatctcctgccagacctcggaacggccacccttgatccattccgcaaggcaaagaccctgaacgttaagttcttcgttcacgatcctttcacccgcgaggcattctcccgcgacccacgcaacgtagcacgcaaggcagagcagtacctggcatccaccggcattgcagacacctgcaacttcggcgccgaggctgagttctacctcttcgactccgttcgctactccaccgagatgaactccggcttctacgaagtagataccgaagaaggctggtggaaccgtggcaaggaaaccaacctcgacggaaccccaaacctgggcgcaaagaaccgcgtcaagggtggctacttcccagtagcaccatacgaccaaaccgttgacgtgcgcgatgacatggttcgcaacctcgcagcttccggcttcgctcttgagcgtttccaccacgaagtcggtggcggacagcaggaaatcaactaccgcttcaacaccatgctccacgcggcagatgatatccagaccttcaagtacatcatcaagaacaccgctcgcctccacggcaaggctgcaaccttcatgcctaagccactggctggcgacaacggttccggcatgcacgctcaccagtccctctggaaggacggcaagccactcttccacgatgagtccggctacgcaggcctgtccgacatcgcccgctactacatcggcggcatcctgcaccacgcaggcgctgttctggcgttcaccaacgcaaccctgaactcctaccaccgtctggttccaggcttcgaggctccaatcaacctggtgtactcacagcgcaaccgttccgctgctgtccgtatcccaatcaccggatccaacccaaaggcaaagcgcatcgaattccgcgctccagacccatcaggcaacccatacctgggcttcgcagcgatgatgatggccggcctcgacggcatcaagaaccgcatcgagccacacgctccagtggacaaggacctctacgaactgccaccagaggaagctgcatccattccacaggcaccaacctccctggaagcatccctgaaggcactgcaggaagacaccgacttcctcaccgagtctgacgtcttcaccgaggatctcatcgaggcgtacatccagtacaagtacgacaacgagatctccccagttcgcctgcgcccaaccccgcaggaattcgaattgtacttcgactgctaa

SEQ ID NO: 20 - protein, 477 aa, Brevibacterium lactofermentum
VAFETPEEIVKFIKDENVEFVDVRFTDLPGTEQHFSIPAASFDADTVEEGLAFDGSSIRGFTTIDESDMNLLPDLGTATLDPFRKAKTLNVKFFVHDPFTREAFSRDPRNVARKAEQYLASTGIADTCNFGAEAEFYLFDSVRYSTEMNSGFYEVDTEEGWWNRGKETNLDGTPNLGAKNRVKGGYFPVAPYDQTVDVRDDMVRNLAASGFALERFHHEVGGGQQEINYRFNTMLHAADDIQTFKYIIKNTARLHGKAATFMPKPLAGDNGSGMHAHQSLWKDGKPLFHDESGYAGLSDIARYYIGGILHHAGAVLAFTNATLNSYHRLVPGFEAPINLVYSQRNRSAAVRIPITGSNPKAKRIEFRAPDPSGNPYLGFAAMMMAGLDGIKNRIEPHAPVDKDLYELPPEEAASIPQAPTSLEASLKALQEDTDFLTESDVFTEDLIEAYIQYKYDNEISPVRLRPTPQEFELYFDC

SEQ ID NO: 21 - DNA, 672 bp, Brevibacterium lactofermentum
  CDS (1)..(669)
atggcaggagcagtgggacgcccccggagatcagctccgcgacgggcaggcaagaatcctcgcgaggagattcttgacgcctctgctgagcttttcacccatcaaggcttcgcaacaacctccacgcatcaaatcgctgatgccgtgggaatccgccaagcctcgctgtattatcacttcccgtctaagacggaaatcttcctcaccctcctgaaatctaccgtcgagccgtccactgtgctcgccgaagacttaagcatcctggatgcaggacctgagatgcgcctctgggcaatcgttgcctccgaagtgcgtctgctgctgtccaccaagtggaacgtcggtcgcctgtaccaactccccatcgttggttctgaagagttcgccgagtaccacagccagcgcgaagccctcaccaacatcttccgcgacctcgccaccgaaatcgtcggtgacgacccccgcgcagaactccccttccacatcaccatgtcggtgatcgaaatgcgtcgcaacgacggcaagattccaagcccgctttccgcagacagcctcccggagaccgcaattatgcttgccgacgcctccctcgccgtcctcggcgcgtcgctgcccgccgaccgggtcgaaaaaacgcttgaactaatcaagcaggctgacgcgaaataacca

SEQ ID NO: 22 - protein, 222 aa, Brevibacterium lactofermentum
MAGAVGRPRRSAPRRAGKNPREEILDASAELFTHQGFATTSTHQIADAVGIRQASLYYHFPSKTEIFLTLLKSTVEPSTVLAEDLSILDAGPEMRLWAIVASEVRLLLSTKWNVGRLYQLPIVGSEEFAEYHSQREALTNIFRDLATEIVGDDPRAELPFHITMSVIEMRRNDGKIPSPLSADSLPETAIMLADASLAVLGASLPADRVEKTLELIKQADAK

SEQ ID NO: 23 - DNA, 2076 bp, Brevibacterium lactofermentum
  CDS (1)..(2076)
atgaataatccagcccagctgcgccaagatactgaaaaggaagtcctggcgttgctgggctctttggttttacccgccggcaccgcgcttgccgccaccggatctttggccaggtccgaactcacgccgtattccgatttggacctcattttgatccatccaccaggggcaaccccggatggcgtggaggatttgtggtacccgatttgggacgcaaaaaagcgcctcgactactccgtgcgcaccccagatgagtgcgtggctatgatttctgcggattccactgcagcccttgccatgcttgacctgcgatttattgctggcgatgaggatctgtgtgccaaaacgcgccggcgcatcgtggagaagtggcgccaggaactcaacaaaaacttcgacgccgttgtggacaccgcgattgcccgttggcgccgctccggacccgtcgtggcaatgacgcggccagatcttaaacacggcaggggagggctgcgcgatttcgaactgatcaaggccctcgcgctcggccacctatgcaacgttccacagctagatacgcaacaccagctgcttctcgacgcccgcaccttgctgcacgtccacgcgcgacgctcccgcgacgtccttgatcccgaatttgcggtggatgtggccatggatttgggctttgttgaccgctatcacttaggccgggagatcgccgatgcagcccgcgccattgatgacggcctgaccaccgcgctggccaccgcccgtggcattttgccacgtcgcacgggttttgcttttaggaatgcttctcgacgcccacttgatcttgatgtcgtcgacgccaacggcactatcgaattgtccaaaaaaccagatcttaatgatcccgcacttccacttcgagtggccgcagccgcagcgaccaccggacttccggtggcagaatcaacctgggctcgacttaatgaatgcccgccacttcctgagccatggcctgccaatgcagcaggggacttctttcggattctctccagtccgaaaaactcacgccgagtggtgaaaaatatggatcgccacggattgtggtcgcgttttgttccagaatgggaccgcatcaaagggcttatgccccgtgaacccagccatatttccaccatcgatgaacatagtctgaacactgttgcaggatgtgcgctagaaactgtgaccgtcgcgcgccccgatcttttagttttgggagccttgtaccacgacattggcaagggcttcccgcgtccacacgaacaagtaggtgcagagatggtggcgagggccgcgagccgcatggggttgaaccttcgcgatcgtgccagcgtgcaaacgctggtcgccgagcacaccgcggtggccaaaatcgccgcgcgccttgatccctcctcggagggcgccgtcgataagctgcttgatgctgttaggtatgacctggtgacattgaatctgcttgaggtgctaacagaagctgatgcgaaagccacggggcctggcgtatggacggcgcgtttggagcatgcgctgcggattgtgtgcaagcgtgcgcgtgatcgcctcaccgatattcgcccggttgcgccgatgattgcgccgcgtagcgaaattggtttggtggaacgcgatggcgtgttcacagtgcaatggcacggcgaagacttacatcggattcttggcgtaatttatgccaaaggatggacaatcaccgcggcgcgcatgctggccaatggtcaatggagtgcggaatttgatgtccgcgcaaacggcccccaagattttgatccgcagcatttcctgcaggcatatcaatccggtgtgttttccgaggttcccattccagcacctgggataacagccacattttggcacgggaacactttagaagtgcgcactgagcttcgcacaggagctatttttgccctgctcagaacattgcccgatgccctctggatcaacgctgtgacccgcggtgcgaccctgattatccaggcagcactgaagcccggcttcgatcgagcaacggtggaacgctccgtagtcaggtcgttggcaggtagc

SEQ ID NO: 24 - protein, 692 aa, Brevibacterium lactofermentum
MNNPAQLRQDTEKEVLALLGSLVLPAGTALAATGSLARSELTPYSDLDLILIHPPGATPDGVEDLWYPIWDAKKRLDYSVRTPDECVAMISADSTAALAMLDLRFIAGDEDLCAKTRRRIVEKWRQELNKNFDAVVDTAIARWRRSGPVVAMTRPDLKHGRGGLRDFELIKALALGHLCNVPQLDTQHQLLLDARTLLHVHARRSRDVLDPEFAVDVAMDLGFVDRYHLGREIADAARAIDDGLTTALATARGILPRRTGFAFRNASRRPLDLDVVDANGTIELSKKPDLNDPALPLRVAAAAATTGLPVAESTWARLNECPPLPEPWPANAAGDFFRILSSPKNSRRVVKNMDRHGLWSRFVPEWDRIKGLMPREPSHISTIDEHSLNTVAGCALETVTVARPDLLVLGALYHDIGKGFPRPHEQVGAEMVARAASRMGLNLRDRASVQTLVAEHTAVAKIAARLDPSSEGAVDKLLDAVRYDLVTLNLLEVLTEADAKATGPGVWTARLEHALRIVCKRARDRLTDIRPVAPMIAPRSEIGLVERDGVFTVQWHGEDLHRILGVIYAKGWTITAARMLANGQWSAEFDVRANGPQDFDPQHFLQAYQSGVFSEVPIPAPGITATFWHGNTLEVRTELRTGAIFALLRTLPDALWINAVTRGATLIIQAALKPGFDRATVERSVVRSLAGS